(19) United States
(12) Patent Application Publication
(10) Pub. No.: US 2017/0020618 A1
(43) Pub. Date: Jan. 26, 2017

(54) Stethoscope Cover Device

(71) Applicant: Carri Lakes, Highland, CA (US)

(72) Inventor: Carri Lakes, Highland, CA (US)

(21) Appl. No.: 15/219,207

(22) Filed: Jul. 25, 2016

Related U.S. Application Data
(60) Provisional application No. 62/196,571, filed on Jul. 24, 2015.

(51) Int. Cl. A61B 46/10; A61B 7/02

(52) U.S. Cl. CPC A61B 46/10; A61B 7/02

(57) ABSTRACT

A stethoscope cover device for covering a portion of a stethoscope is provided. The stethoscope has a chest piece and lower tubing immediately adjacent the chest piece. The stethoscope cover device comprises a bulb-shaped sleeve having a bulbous portion and a cylindrical portion. An opening is formed in the cylindrical portion. The chest piece and lower tubing of the stethoscope are receivable within the sleeve through the opening The sleeve creates a barrier eliminating the stethoscope as a source of hospital acquired infections.

### CLAIM OF PRIORITY

This patent application claims priority under 35 USC 119 (e) (1) from U.S. Provisional Patent Application Se. No. 62/196,571 filed Jul. 24, 2015, of common inventorship herewith entitled, “Care Cover,” which is incorporated herein by reference as though the same were set forth in its entirety.

### FIELD OF THE INVENTION

The present invention pertains to the field of medical supplies, and more specifically to the field of stethoscope covers.

### BACKGROUND OF THE INVENTION

It has become a commonplace for a patient, who is going into a hospital or healthcare facility for a procedure, to have two worries. The first worry is recovering from one's existing injury or illness and the second worry is picking up a new infection or illness while in the hospital. Hospital acquired infections, known in the medical field as HAIs or nosocomial infections, are infections that patients acquire during hospital stays. Bacterial, viral or fungal infections are spread from contaminated surfaces to a patient, or from one patient to another patient. Urinary tract infections, surgical site infections, bloodstream infections, and pneumonia are common types of HAIs which are caused by pathogens that easily spread through a body. Hospitals are becoming particularly notorious for spreading potentially lethal infections. Many hospital patients have compromised immune systems, so they are less able to fight off infections.

Transferring infections occurs due to poor conditions at a hospital or a healthcare facility. HAIs occur due to medical staff not following proper hygienic procedures such as washing their hands with an antibacterial scrub before seeing each patient or wearing fresh examination gloves for each patient. Pathogens are spreadable from patient to patient by instruments frequently used by medical staff. Medical personnel who work in Intensive Care and Critical Care often are required to wear gowns, gloves, masks and protective eyewear to protect themselves and their patients, and to prevent the transmission of infection between patients. This protective equipment is single use only and disposable.

Used by Physicians, RNs, Respiratory Therapists, Speech Therapists and other medical professionals, the stethoscope is a standard amplifying instrument accessed multiple times during a shift to assess a patient's medical condition. Disposable stethoscopes are provided for use in intensive and critical units, but they are cheaply made and difficult to use in obtaining an accurate assessment on these critical patients. Different persons often use the same disposable stethoscope and neglect to wipe it down after use. So medical staff often use their own stethoscopes in the patient's room. Bleach wipes are available and required for the disinfection of equipment that comes out of an isolation room, so when medical staff use their own stethoscopes, they wipe them down with bleach upon leaving the room. After time, the bleach ruins expensive stethoscopes and medical professionals must buy a new one. The surface must remain wet with bleach solution for at least four minutes to be effective in killing certain organisms. The wipe must be sufficiently saturated with liquid in order to ensure that this requirement is met. When finished, if the bleach isn't completely dry, it can bleach one's clothes, ruining a uniform top, and irritate the skin on one's neck. To avoid this problem, medical staff use a disposable glove to cover the chest piece or bell and lower tubing of the stethoscope, keeping the stethoscope from touching the patient's bare skin. The glove provides a very thin barrier, making it possible to hear what is being assessed while maintaining isolation precautions.

Using gloves as a protective cover works, but is very awkward with five finger protrusions bunched at one end.

The prior art has put forth several designs for stethoscope covers. Among these are:

U.S. Pat. No. 8,662,244 to Kenneth I. Fishberger and David M. Schwartzberg describes a cover for a stethoscope having a head and a diaphragm is provided. The cover includes a first region having first and second sides, and a second region configured to be continuous with the first region and including a crimpable material to form fit around at least the head of the stethoscope so as to cover the head of the stethoscope from contamination. The first side includes adherent material for removably adhering the first region to the diaphragm of the stethoscope and the second side includes an antimicrobial layer including an antimicrobial substance that can neutralize or destroy microbes disposed thereon.

U.S. Pat. No. 8,042,646 to John David Gross describes a cover for a stethoscope to prevent microbes from transferring from a patient to a stethoscope. In one embodiment, a stethoscope cover comprises an antimicrobial barrier having a first and second surface, image forming material disposed on the antimicrobial barrier first surface, and an adhesive layer disposed on the second surface of the antimicrobial barrier to attach the antimicrobial layer to a stethoscope. In some embodiments, the image forming material is configured to depict an image of a product. In some embodiments, the image forming material is configured to depict an image of an animal or character to evoke feelings of happiness in a young patient.

U.S. Pat. No. 7,823,690 to Bruce Hirsch and David Miller describes a disposable stethoscope cover that covers at least the surface of the diaphragm. The cover contacts a patient during use and reduces transmittal of microorganisms. The cover is in the form of a flexible sheet having a top and a bottom surface. The sheet has an adhesive material on a portion of the surface thereof that secures the sheet to the surface of the diaphragm. The sheet further includes at least one wing extending from the sheet. The wing has an adhesive on at least one surface thereof for securing the wing to the sheet when the wing is folded over at least a portion of a tube section of the stethoscope.

None of these prior art references describe the present invention.

### SUMMARY OF THE INVENTION

It is an object of the present invention to provide a onetime use, disposable hygienic cover that is placed over a chest piece and lower tubing of a stethoscope.

The present invention is a stethoscope cover device for covering a portion of a stethoscope. The stethoscope has a chest piece and lower tubing immediately adjacent the chest piece. The stethoscope cover device comprises a bulb-shaped sleeve having a bulbous portion and a cylindrical portion. An opening is formed in the cylindrical portion. The chest piece and lower tubing of the stethoscope are receivable within the sleeve through the opening. The sleeve creates a barrier eliminating the stethoscope as a source of hospital acquired infections.

In addition, the present invention further includes a method for covering a portion of a stethoscope. The stethoscope has a chest piece and lower tubing immediately adjacent the chest piece. The method comprises providing a bulb-shaped sleeve having a bulbous portion and a cylindrical portion, forming an opening in the cylindrical portion, inserting the chest piece and lower tubing of the stethoscope into the sleeve through the opening, and creating a barrier eliminating the stethoscope as a source of hospital acquired infections.

The present invention further includes a stethoscope cover device for covering a portion of a stethoscope. The stethoscope has a chest piece and lower tubing immediately adjacent the chest piece. The stethoscope cover device comprises a bulb-shaped sleeve having a bulbous portion and a cylindrical portion. An opening is formed in the cylindrical portion. The chest piece and lower tubing of the stethoscope are receivable within the sleeve through the opening. The sleeve is constructed from a deformable, resilient material. The sleeve deforms over the chest piece and the lower tubing and springs back and/or rebounds toward an original shape thereby snugly encasing the chest piece and the lower tubing immediately adjacent the chest piece. The sleeve creates a barrier eliminating the stethoscope as a source of hospital acquired infections.

### BRIEF DESCRIPTION OF THE DRAWINGS

FIG. 1 is a perspective view illustrating a stethoscope cover device, constructed in accordance with the present invention, showing the stethoscope cover device having a shape constructed to cover the chest piece and lower tube of the stethoscope.

FIG. 2 is a perspective view illustrating the stethoscope cover device, constructed in accordance with the present invention, showing the chest piece end of the stethoscope being inserted into the stethoscope cover device.

FIG. 3 is a perspective view illustrating the stethoscope cover device, constructed in accordance with the present invention, showing the chest piece and lower tube of the stethoscope completely covered by the stethoscope cover device.

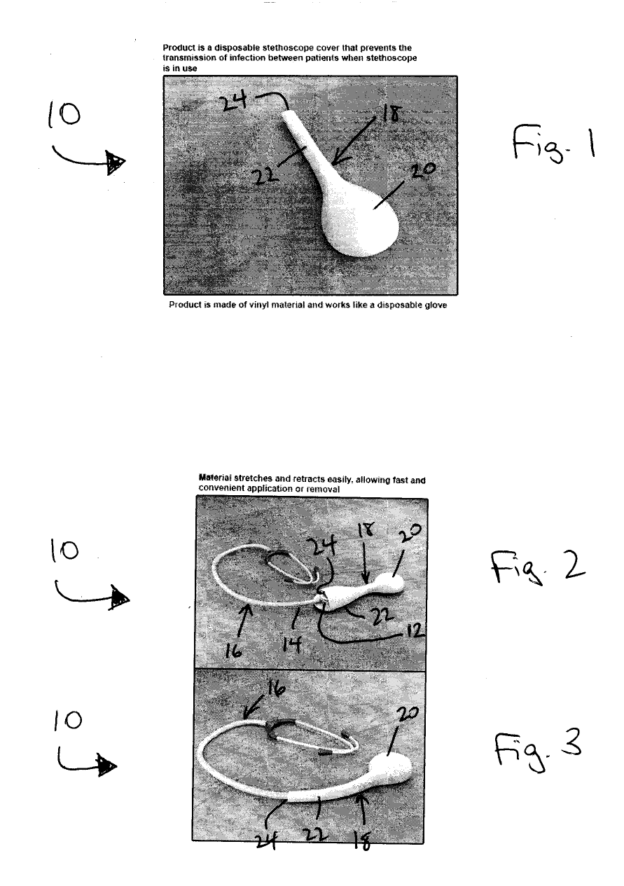

### DETAILED DESCRIPTION OF THE INVENTION

The present invention, hereinafter referred to as the Stethoscope Cover Device, indicated generally at 10, is a onetime use, disposable hygienic cover placed over a chest piece 12 or bell and lower tubing 14 immediately adjacent the chest piece 12 of a stethoscope 16. Just as fresh medical exam gloves are worn by medical personnel to prevent the transfer of bacteria, body fluids, fungi and viruses by the hands of those involved in patient care, the Stethoscope Cover Device 10 provides a protective barrier between the stethoscope 16 and a patient. A fresh Stethoscope Cover Device 10 is used with each patient to create a barrier between the stethoscope 16 and the patient's skin, eliminating the stethoscope 16 as a source of hospital acquired infections.

The Stethoscope Cover Device 10 of the present invention is a bulb-shaped sleeve 18 having a bulbous portion 20 sized and shaped for receiving the chest piece 12 of the stethoscope 16 and a cylindrical portion 22 sized and shaped for receiving at least a portion of the lower tubing 14 of the stethoscope 16. The cylindrical portion 22 has an opening 24 for receiving the chest piece 12 and the lower tubing 14, as will be described below. Besides the opening 24, no other openings, apertures, or breaches are present in the sleeve 18.

The bulbous portion 20 and the cylindrical portion 22 of the sleeve 18 of the Stethoscope Cover Device 10 of the present invention are preferably formed from the same material with the bulbous portion 20 and the cylindrical portion 22 of the sleeve 18 being integral to each other. It should be noted that having the bulbous portion 20 and the cylindrical portion 22 constructed from different materials that are connected together is within the scope of the present invention. In addition, the material of the sleeve 18 positioned at the front of the chest piece 12 can be thinner or constructed from a different material to allow better sound transfer therethrough.

The sleeve 18 of the Stethoscope Cover Device 10 of the present invention is preferably constructed from a medical grade, latex free material such as vinyl. Regardless of the material used for the sleeve 18, it is desirable for the material to preferably be a deformable, resilient material allowing the sleeve 18 to deform over the chest piece 12 and the lower tubing 14 and spring back and/or rebound toward an original shape thereby snugly encasing the chest piece 12 and the lower tubing 14 immediately adjacent the chest piece 12.

The Stethoscope Cover Device 10 of the present invention preferably measures approximately six inches in length and two inches in diameter at its widest point, i.e., the bulbous portion 20. Like examination gloves, the Stethoscope Cover Device 10 is preferably dispensed from multiunit boxes, so that patient care personnel entering a work area simply remove a fresh Stethoscope Cover Device 10 from the box dispenser and pull it over the chest piece 20 and at least a portion of the lower tubing 22 of their stethoscope 16. The medical professionals then grip their stethoscopes 16 by the covered chest piece 20 and covered lower tubing 22 and proceed with their patient examination. The Stethoscope Cover Device 10 is removed and disposed of following the examination. A fresh Stethoscope Cover Device 10 is employed with each patient. The Stethoscope Cover Device 10 accommodates all sizes and shapes of stethoscopes 16 currently in use, and are easy to apply and remove.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claim.

1. A stethoscope cover device for covering a portion of a stethoscope, the stethoscope having a chest piece and lower tubing immediately adjacent the chest piece, the stethoscope cover device comprising:
a bulb-shaped sleeve having a bulbous portion and a cylindrical portion;
an opening formed in the cylindrical portion;
wherein the chest piece and lower tubing of the stethoscope are receivable within the sleeve through the opening; and
wherein the sleeve creates a barrier eliminating the stethoscope as a source of hospital acquired infections.

2. The stethoscope cover device of claim 1 wherein the sleeve is a onetime use, disposable hygienic cover positionable over the chest piece and lower tubing of the stethoscope.

3. The stethoscope cover device of claim 1 wherein the sleeve has no additional openings, apertures, and breaches.

4. The stethoscope cover device of claim 1 wherein the bulbous portion is capable of receiving the chest piece of the stethoscope and the cylindrical portion is capable of receiving the lower tubing of the stethoscope.

5. The stethoscope cover device of claim 1 wherein the bulbous portion and the cylindrical portion of the sleeve are constructed from the same material with the bulbous portion and the cylindrical portion of the sleeve being integral to each other.

6. The stethoscope cover device of claim 1 wherein the bulbous portion and the cylindrical portion are constructed from different materials that are connected together.

7. The stethoscope cover device of claim 1 wherein the sleeve is constructed from a medical grade, latex free vinyl material.

8. The stethoscope cover device of claim 1 wherein the sleeve is constructed from a deformable, resilient material allowing the sleeve to deform over the chest piece and the lower tubing and spring back and/or rebound toward an original shape thereby snugly encasing the chest piece and the lower tubing immediately adjacent the chest piece.

9. The stethoscope cover device of claim 1 wherein the sleeve is dispensed from multiunit boxes.

10. A method for covering a portion of a stethoscope, the stethoscope having a chest piece and lower tubing immediately adjacent the chest piece, the method comprising:
providing a bulb-shaped sleeve having a bulbous portion and a cylindrical portion;
forming an opening in the cylindrical portion;
inserting the chest piece and lower tubing of the stethoscope into the sleeve through the opening; and
creating a barrier eliminating the stethoscope as a source of hospital acquired infections.

11. The method of claim 10 wherein the sleeve has no additional openings, apertures, and breaches.

12. The method of claim 10 and further comprising:
constructing the bulbous portion and the cylindrical portion from the same material with the bulbous portion and the cylindrical portion of the sleeve being integral to each other.

13. The method of claim 10 and further comprising:
constructing the sleeve from a deformable, resilient material allowing the sleeve to deform over the chest piece and the lower tubing and spring back and/or rebound toward an original shape thereby snugly encasing the chest piece and the lower tubing immediately adjacent the chest piece.

14. A stethoscope cover device for covering a portion of a stethoscope, the stethoscope having a chest piece and lower tubing immediately adjacent the chest piece, the stethoscope cover device comprising:
a bulb-shaped sleeve having a bulbous portion and a cylindrical portion;
an opening formed in the cylindrical portion;
wherein the chest piece and lower tubing of the stethoscope are receivable within the sleeve through the opening;
wherein the sleeve is constructed from a deformable, resilient material;
wherein the sleeve deforms over the chest piece and the lower tubing and springs back and/or rebounds toward an original shape thereby snugly encasing the chest piece and the lower tubing immediately adjacent the chest piece; and
wherein the sleeve creates a barrier eliminating the stethoscope as a source of hospital acquired infections.

15. The stethoscope cover device of claim 14 wherein the sleeve is a onetime use, disposable hygienic cover positionable over the chest piece and lower tubing of the stethoscope.

16. The stethoscope cover device of claim 14 wherein the sleeve has no additional openings, apertures, and breaches.

17. The stethoscope cover device of claim 14 wherein the bulbous portion and the cylindrical portion of the sleeve are constructed from the same material with the bulbous portion and the cylindrical portion of the sleeve being integral to each other.

18. The stethoscope cover device of claim 14 wherein the bulbous portion and the cylindrical portion are constructed from different materials that are connected together.

19. The stethoscope cover device of claim 14 wherein the sleeve is constructed from a medical grade, latex free vinyl material.

20. The stethoscope cover device of claim 14 wherein the sleeve is dispensed from multiunit boxes.
